(19) United States
(12) Patent Application Publication
(10) Pub. No.: US 2026/0090614 A1
(43) Pub. Date: Apr. 2, 2026

(54) ADJUSTABLE NON-CONDUCTIVE SMART RING WITH UNIVERSAL FINGER FIT FOR HEALTH MONITORING AND CONTACTLESS TRANSACTIONS

(71) Applicants: Pulkit Ahuja, Gurgaon (IN); Deeksha Raheja, Gurgaon (IN)

(72) Inventor: Pulkit Ahuja, Gurgaon (IN)

(21) Appl. No.: 19/352,524

(22) Filed: Oct. 8, 2025

(30) Foreign Application Priority Data
Oct. 8, 2024 (IN) 202411076093

(51) Int. Cl. A44C 9/00; A61B 5/00; A61B 5/024

(52) U.S. Cl. CPC A44C 9/0053; A61B 5/02427; A61B 5/746

(57) ABSTRACT

The disclosure relates to an adjustable non-conductive smart ring (100) wearable on a finger, including a ring body (102), one or more electronic modules (106) with a biometric sensor (108) and wireless interface (110), and an adjustment mechanism (112) that varies a finger opening (104) to provide reversible resizing. The mechanism may include a rotatable dial (114) with screw (116) and insert (118), hinged halves (120a, 120b) with latch (122), or a segmented gear-drive (126). Certain embodiments use an actuator (210) and controller (212) to automatically adjust fit based on a biometric metric, while a safety-release system (312) with break-away interface (314) or safe-cut indicator (318) allows emergency removal. The smart ring maintains non-conductive safety, biometric accuracy, and one-size adaptability for health monitoring, contactless payment, and industrial use.

### FIELD OF THE EMBODIMENTS

The present disclosure relates generally to smart wearable devices, particularly to an adjustable non-conductive ring for use in healthcare, safety, and financial technology. More specifically, the disclosure pertains to a finger-worn device that configured to dynamically adjust its size to fit different fingers while being made of electrically non-conductive materials for safe use by industrial workers. The ring further integrates electronic modules for health monitoring, geolocation tracking, and contactless payments, serving as a versatile wearable for fitness, safety, and financial transactions.

### BACKGROUND OF THE EMBODIMENTS

Wearable rings have long been used as accessories and status symbols. In recent years, smart rings have emerged that pair with mobile devices to display health data (e.g., heart rate, blood oxygen level) and even enable payment transactions. However, existing smart rings typically come in fixed sizes, which creates several practical challenges. Procuring the correct size is often a long and cumbersome process—users must measure their finger, order a specific size, or use trial kits with multiple rings to find a proper fit. This process is inconvenient for both buyers and retailers, requiring the stocking of many size variants and resulting in logistical inefficiencies.

Moreover, over time a person's finger size may change due to weight fluctuations, temperature variations, or physiological factors, rendering a once-perfect ring either uncomfortably tight or too loose. In such cases, the user must repeat the entire sizing and purchasing process. There is therefore a clear need for a one-size-fits-all smart ring that may adapt to the wearer's finger dynamically, eliminating sizing guesswork and reducing inventory and waste.

Another concern with existing rings (including smart rings) is safety for blue-collar and industrial workers. Many workplaces prohibit metal jewelry on the hands due to the risk of electrocution or snagging injuries. A conductive metal ring may accidentally touch live electrical equipment or become caught in moving machinery, causing serious harm. According to OSHA guidelines, wearing metal rings is often not permitted in such environments. Workers, however, still require the benefits of health monitoring, location tracking, and communication devices, but in a form factor that is non-conductive and may be safely broken away or quickly removed in emergencies to prevent injury.

At the same time, the importance of real-time health monitoring has grown significantly for both personal wellness and occupational safety. For example, a worker operating in high temperatures or under heavy physical exertion may face risks such as dehydration, heat stroke, or overexertion. Continuous monitoring of metrics like heart rate, body temperature, or blood oxygen level may allow early detection of health anomalies and timely intervention. In addition, location tracking of personnel on worksites enhances overall safety and accountability.

The rise of contactless payment technologies, such as NFC and UPI, also presents an opportunity for rings to serve as convenient payment and authentication tokens, further extending their utility beyond fitness tracking.

In a nutshell, there exists a need for an adjustable smart ring that integrates multiple features:
One-size-fits-all adjustability, to simplify sizing and enable long-term use despite changes in finger dimensions;
Non-conductive construction, ensuring safe use in industrial and high-risk environments by reducing electrical and mechanical hazards;
Health and fitness sensors, for continuous monitoring of vital parameters; and
Communication and payment modules, such as NFC and GPS, to extend functionality beyond basic health tracking.

Such a device would enable organizations to equip their workforce with a single, universally fitting model that provides health and safety telemetry, while also allowing consumers to wear a single smart ring for fitness, payment, and daily use without concerns about sizing or safety.

Recent, widely reported incidents underscore a critical safety gap in battery-powered smart rings: unlike watches or bands, rings do not unbuckle. Consequently, any finger swelling or internal battery swelling may trap the wearer's finger and quickly compromise circulation.

In late September-early October 2025, multiple outlets reported that a Samsung Galaxy Ring user experienced in situ battery swelling while preparing to board a flight. The ring tightened painfully, became irremovable, and the traveler was denied boarding; hospital staff ultimately removed the device using ice and medical lubricant. Samsung described the event as extremely rare and advised that, in last-resort cases, removal should follow a specific cut orientation to avoid the embedded battery highlighting how emergency removal is non-trivial in sealed rings with internal cells.

These reports, along with community documentation of similar battery health anomalies, demonstrate that current arts may impede rapid removal and risk circulatory compromise when either the finger or the battery enclosure swells, particularly where a rigid outer frame offers little outward compliance.

Accordingly, there remains a pressing need for finger-worn devices that integrate mechanical and software fail safes including on-demand decompression, quick-release or breakaway interfaces, safe-cut guidance, and auto-loosen control—so that even under internal battery faults or environmental stressors, the ring may rapidly relieve constriction and be safely removed without endangering the wearer.

Several ring and wearable mechanisms have been developed previously, each addressing partial aspects of comfort, wearability, or sensing, but failing to provide a comprehensive, safe, and dynamically adjustable smart ring as described herein.

Fixed-Size Smart Rings: Existing smart rings incorporate biometric sensors such as heart rate, blood oxygen, and motion sensors, and in some cases contactless payment modules. However, these devices typically employ rigid metallic or conductive housings that are manufactured in discrete, non-adjustable sizes. Users must rely on trial or sizing kits to determine the correct fit prior to purchase, which leads to inventory complexity, procurement delays, and material waste. Such rings offer no adaptability to physiological changes in finger size over time, and their conductive structure poses safety hazards for users in industrial or high-voltage environments. Existing arts also lack mechanical adjustability and emergency removal features.

Elastic or Stretchable Rings: Some wearable rings and accessories achieve limited flexibility through the use of elastic silicone bands or stretchable polymer inserts. These rely solely on the inherent elasticity of the material, providing no active control over the degree of fit. Prolonged use leads to material fatigue, loss of tension, and inconsistent fit accuracy. Moreover, such rings cannot house rigid or sensitive electronic assemblies, such as batteries, circuit boards, or antennas, because continuous stretching would compromise electronic integrity. As a result, these rings are unsuitable for advanced smart applications such as health monitoring, location tracking, or digital payments.

Split Rings and Ratchet-Type Adjustable Rings: Conventional jewelry and utility rings have long utilized split-band, ratchet, or telescopic mechanisms to provide limited manual resizing. These typically employ sliding, screw, or latch elements that allow the circumference to be adjusted by small increments. However, such mechanisms are:
Conductive, and therefore unsafe for industrial or electrical applications;
Purely mechanical, with no integration of electronic modules or health sensors;
Bulky and externally exposed, reducing wearer comfort and aesthetic appeal; and
Unsuitable for dynamic or automated adjustment, since they depend entirely on manual operation.

Furthermore, such arts lack any emergency release or pressure-relief mechanism, making them unsafe if the ring or the wearer's finger swells.

Adjustable Wearable Bands and Wrist Devices: Some wearable wrist devices employ motorized adjusters, micro-actuators, or flexible strap systems to automatically vary band tension for improved comfort or sensing accuracy.

While such systems demonstrate controlled fit adjustment, they operate on large, flexible surfaces and are not directly applicable to rigid, finger-worn structures. The geometric and safety constraints of a closed ring differ fundamentally from those of wristbands, which can be easily unclasped in emergencies. Thus, existing adjustable wearables cannot be miniaturized or adapted to provide safe, automated fit control in a ring form factor.

Modular and Detachable Smart Rings: Certain prior smart rings have explored detachable or modular electronic components, enabling the user to replace or recharge specific modules. However, these modular systems focus on component interchangeability, not on dynamic size adjustment or safety during wear. They continue to rely on fixed metallic structures and offer no adaptive or breakaway mechanisms for fit or emergency release.

Rings with Internal Sensors or Rotating Sections: Some prior devices incorporate rotatable or repositionable elements for input control, gesture detection, or data transmission. While mechanically interesting, these mechanisms do not alter the inner diameter of the ring, nor do they contribute to fit comfort or safety. They remain static in size and rigid in structure, addressing only user interaction, not ergonomic adaptability.

Dynamic-Fit Wearables Using Actuators or Tensioning Systems: Certain research prototypes and wearable concepts disclose actuator-based or tensioning systems for automatically adjusting fit, primarily for wristbands or headgear. These arts utilize motors, linear actuators, or inflatable bladders to control tightness around larger body parts. However, these systems are bulky, energy-intensive, and not feasible within the limited volume of a ring form factor. Additionally, they do not address safe decompression or fail-safe removal when electrical or thermal faults occur.

Battery-Powered Rings without Safety Mechanisms: Prior battery-integrated smart rings have been observed to exhibit thermal swelling, deformation, or finger entrapment under certain conditions due to sealed, rigid enclosures. Such arts do not provide a defined cut path, decompression vent, or breakaway seam to enable safe removal during swelling or failure. There is no disclosure of a “safe-cut guide,” sacrificial seam, or non-conductive release interface that ensures protection of the wearer and battery components during emergency removal.

Absence of Safe Mechanical Adjustment and Control Mechanisms: Existing smart rings and wearable devices do not incorporate user-operable hinge-and-pin latches, strap-based breakaway joints, or shearable connecting elements that permit safe mechanical release during emergencies. Known arts rely on rigid, continuous frames with no provision for controlled separation or decompression under swelling or thermal expansion. Similarly, existing adjustable mechanisms—such as screws, dials, or ratchets—are manually operated, metallic, and externally exposed, and they lack sealed, non-conductive arts capable of maintaining position through power-off mechanical locking. Moreover, prior devices do not include any adaptive control systems that dynamically regulate fit based on sensor feedback, biometric parameters, or environmental conditions.

From the foregoing, it is evident that known devices fail to:
Provide continuous or user-independent dynamic fit adjustment for varying finger sizes;
Integrate non-conductive materials with embedded electronics in a compact, safe form factor;
Include emergency release, decompression, or safety-cut provisions;
Combine health monitoring, location tracking, and payment capabilities in a universal-fit configuration; or
Offer automated feedback-based fit control ensuring both comfort and safety.

Therefore, there exists a need to address the aforementioned shortcomings (e.g., user-unlocked hinge-and-pin latches, strap-based or shearable elements, dial/screw mechanisms with power-off locking, and adaptive control), providing multiple independent pathways to restore circulation and enable safe emergency removal under fault conditions.

### OBJECTIVE OF THE EMBODIMENTS

The primary object of the present disclosure is to provide an adjustable non-conductive smart ring device that may dynamically adapt its size to fit different finger dimensions, thereby offering a universal one size fits all wearable solution for health, safety, and financial applications.

Another object of the present disclosure is to provide a safe and ergonomic ring configuration constructed from non-conductive materials, enabling its use by industrial and field workers in electrically hazardous or high-risk environments.

A further object of the present disclosure is to provide a mechanically adjustable structure incorporating user unlocked hinge and pin latches, strap based or shearable connecting elements, and dial or screw-based adjusters configured to permit manual fit adjustment or rapid release when required.

Another object of the present disclosure is to provide electromechanical mechanisms including micro motors, actuators, or shape memory materials that enable automatic or semi-automatic fit control based on sensor feedback, temperature, pressure, or biometric parameters.

It is another object of the present disclosure to provide power off locking systems that may retain the adjusted size and position even when the electrical subsystem is inactive, ensuring both energy efficiency and fail-safe safety performance.

A further object of the present disclosure is to provide emergency decompression and removal features including sacrificial seams, breakaway bridges, or safe cut indicators that guide safe manual or tool assisted removal of the ring under swelling, overheating, or electrical fault conditions without causing injury or damaging embedded electronics.

Yet another object of the present disclosure is to provide adaptive software control implementing control logics such as AutoFit and SafetyLoosen which monitor biometric and environmental data in real time to dynamically regulate the ring's fit and comfort level.

An additional object of the present disclosure is to provide a method for dynamically adjusting the ring fit comprising detecting biometric or environmental parameters such as heart rate, temperature, or pressure determining deviation from a predefined comfort threshold and actuating a mechanical or electromechanical adjustment to maintain safe and optimal fit.

Another object of the present disclosure is to provide a method for emergency removal of the smart ring wherein activation of a manual control or automatic safety trigger causes the release or disengagement of one or more structural elements such as hinge pins, strap connectors, or shearable joints thereby restoring circulation and enabling rapid safe removal.

It is yet another object of the present disclosure to provide a system comprising the smart ring device an external controller or mobile application and communication interfaces such as Bluetooth NFC or Internet connectivity that enable data synchronization user alerts firmware updates and transaction authentication.

A further object of the present disclosure is to provide a computer implemented method for controlling ring fit and safety features wherein a processor executes stored instructions to perform sensor data acquisition threshold evaluation and actuator control logic ensuring adaptive fit management without continuous user input.

Another object of the present disclosure is to provide a multi-functional smart ring device integrating biometric sensors location tracking and contactless payment functionality within a compact non-metallic form factor capable of continuous operation without compromising safety or comfort.

A still further object of the present disclosure is to provide a universal fit wearable configuration that simplifies manufacturing logistics and workforce deployment eliminating the need for multiple size variants and reducing cost material waste and inventory management complexity.

It is also an object of the present disclosure to provide a robust water resistant and aesthetically refined configuration that ensures durability comfort and reliable operation across varied user environments including industrial fitness and daily lifestyle scenarios.

### SUMMARY OF THE EMBODIMENTS

The present disclosure relates to a class of adjustable non-conductive smart rings that enable dynamic or selective resizing while performing biometric monitoring and wireless communication functions. The disclosure particularly addresses the limitations of existing fixed-size or metallic rings by providing compact mechanical and electromechanical arrangements that permit safe adjustment of the ring opening, real-time biometric sensing, and rapid removal in emergencies without risk of electrical conduction or injury.

In one embodiment, a manually adjustable smart ring (100) is provided. The ring comprises a non-conductive body (102) wearable on a finger and defining a finger opening (104). One or more electronic modules (106) including a biometric sensor (108) and wireless interface (110) are associated with the ring body. A mechanical adjustment mechanism (112) provides continuous and reversible resizing of the finger opening (104) while maintaining a uniform circular geometry and consistent fit. The adjustment mechanism may include a rotatable dial assembly (114) coupled to a threaded screw (116) and crescent-shaped insert (118), a hinged dual-half assembly (120) with latch (122) or pin (124), or a segmented gear-drive assembly (126) having inter-meshing segments (127) actuated by a dial (130). All user-touchable parts (134) are formed of insulating materials, ensuring electrical safety. This embodiment allows one-size-fits-all usability and long-term comfort while eliminating conductive hazards in industrial environments.

In another embodiment, an automatically adjustable smart ring (200) is disclosed. The ring includes a non-conductive body (202), biometric sensors (206), a size-adjustment system (208), an actuation unit (210), and a controller (212). The controller (212) processes signals from the sensors (206) to derive a fit-quality metric and operates the actuation unit (210) until a target value is reached. The size-adjustment system (208) may employ one or more of an electromechanical micro-motor drive (214), a shape-memory element (216), an electro-active polymer assembly (218), or a micro-fluidic membrane (220). The controller (212) enforces torque and temperature limits, activates a safety-loosen mode (222a) when physiological or thermal parameters exceed thresholds, and uses a locking feature (222) to retain position without continuous power. This embodiment introduces intelligent auto-fit control that maintains biometric accuracy and comfort while preventing over-tightening or circulation blockage.

A further embodiment provides a safety-release smart ring (300) having a non-conductive body (302) with electronic modules (306) including a power source (308) and circuit components (310). The ring incorporates a safety-release system (312) that permits controlled parting of the ring under stress or emergency conditions. The system (312) may include a break-away interface (314) that releases above a calibrated threshold (316), a safe-cut indicator (318) aligned with a sacrificial seam (322) having a reduced-thickness web (324) forming a battery keep-out corridor (326), and an emergency-guidance subsystem (328) comprising a lighting element (328a) and controller (328b) to assist visual identification during removal. Additional safety elements include crack-stop fillets (332), insulating shields (338), and spatial separation (336) between conductors (334) and the seam (322). This embodiment ensures safe mechanical or guided tool-based removal of the ring without intersecting internal power components, thereby preventing injury or electrical hazard.

In yet another embodiment, a method of operating an adjustable smart ring (450) is described. The method includes acquiring biometric signals from sensors (452), processing the signals in a controller (454) to compute a fit-quality metric, actuating a size-adjustment system (456) through an actuation unit (458) until the metric attains a target value, and de-energising the actuation unit (458) while a mechanical lock (460) maintains the position. The controller (454) monitors physiological or thermal parameters and initiates a safety-loosen operation (462) when thresholds are exceeded, relieving circumferential pressure. The method further includes enforcing torque and temperature limits, generating alert signals (466), and activating a guidance element (464) to illuminate a safe-cut indicator when a fault signal (468) or emergency input is detected. This method integrates biometric feedback, adaptive mechanical control, and emergency guidance, ensuring optimal fit, user comfort, and fail-safe operation.

Collectively, the embodiments described herein provide a family of smart rings that combine mechanical adaptability, biometric intelligence, and safety assurance. The embodiments address long-standing issues of ring sizing, industrial electrical safety, and emergency removal in battery-powered wearables. The disclosure therefore advances the art by introducing both manual and automated adjustment mechanisms, integrated fail-safe architecture, and responsive operational methods suitable for healthcare, fitness, occupational safety, and contactless transaction applications.

### BRIEF DESCRIPTION OF THE DRAWINGS OF THE EMBODIMENTS

Other objects, features, and advantages of the embodiment will be apparent from the following description when read with reference to the accompanying drawings. In the drawings, wherein like reference numerals denote corresponding parts throughout the several views:

FIG. 1A illustrates a perspective view of a manually adjustable smart ring (100) having a non-conductive ring body (102), a finger opening (104), and electronic modules (106) including a biometric sensor (108) and wireless-communication interface (110).

FIG. 1B shows the rotatable dial assembly (114) with threaded screw (116) and crescent-shaped insert (118) forming part of the adjustment mechanism (112) for varying the diameter of the finger opening (104).

FIG. 1C depicts a hinged dual-half assembly (120) with arcuate halves (120a, 120b) joined by a hinge (121) and secured by a latch (122) or spring-biased pin (124) to provide manual resizing.

FIG. 1D represents a segmented gear-drive assembly (126) having inter-meshing grooves (128), ridges (129), and a rotatable gear dial (130) engaging rack teeth (132) to achieve radial movement of ring segments (127).

FIG. 2A illustrates an automatically adjustable smart ring (200) including biometric sensors (206), a size-adjustment system (208), an actuation unit (210), and a controller (212) for closed-loop fit control.

FIG. 2B shows alternative actuation systems of the smart ring (200), including a micro-motor drive (214), shape-memory element (216), electro-active-polymer layer (218), and micro-fluidic membrane (220).

FIG. 2C depicts a schematic of the controller (212) implementing torque and temperature limits, safety-loosen mode (222a), and locking feature (222b, 222c) for maintaining the target fit.

FIG. 3A illustrates a safety-release smart ring (300) having a ring body (302), safety-release system (312), break-away interface (314), and safe-cut indicator (318) aligned with a sacrificial seam (322).

FIG. 3B shows internal structural details of the seam (322) including a reduced-thickness web (324), crack-stop fillets (332), non-conductive shield (338), and clearance (336) from conductors (334) and power source (308).

FIG. 3C illustrates an emergency-guidance subsystem (328) having lighting element (328a), controller (328b), and optional vibration element (328c) for locating the seam (322) under low-visibility conditions.

FIG. 4A depicts a flow diagram of the method of operating an adjustable smart ring (450), showing acquisition of biometric signals (452), processing by controller (454), actuation of a size-adjustment system (456), and retention of fit by mechanical lock (460).

FIG. 4B illustrates the initiation of a safety-loosen operation (462) when monitored parameters exceed defined thresholds and activation of a guidance element (464) or alert signal (466) to assist emergency removal, with illumination of a safe-cut indicator responsive to a fault signal (468).

### DETAILED DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The disclosure describes an adjustable non-conductive smart ring (100) having a non-conductive ring body (102) that surrounds a finger opening (104) and houses one or more electronic modules (106) including biometric sensors (108), a wireless communication interface (110), and a size adjustment mechanism (112). The ring (100) is intended to provide reversible, comfortable resizing while maintaining electrical isolation and accommodating embedded electronics.

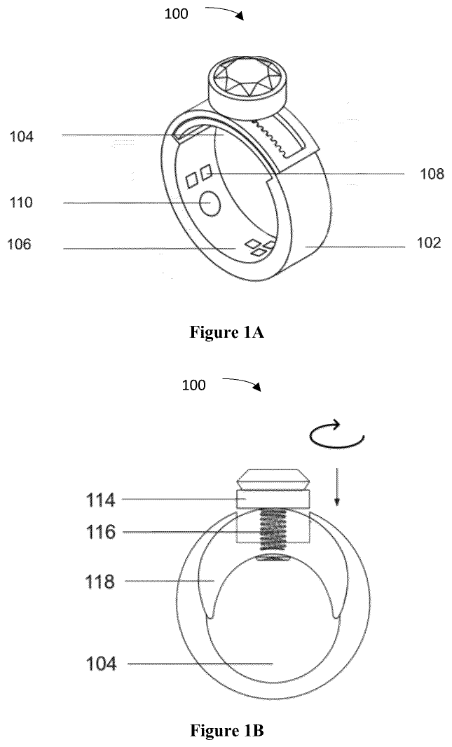

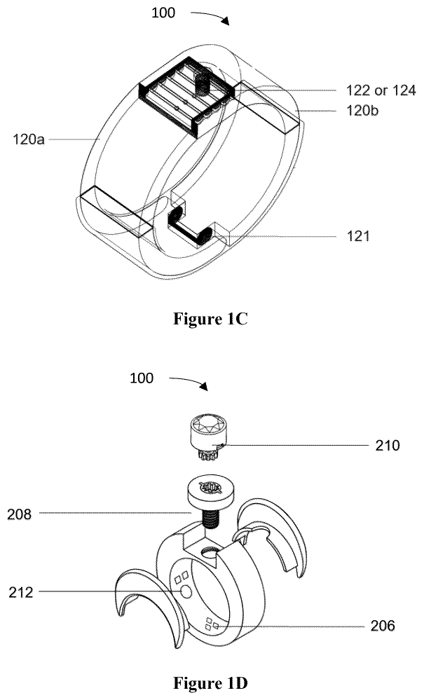

In an embodiment shown in FIG. 1A, the ring (100) incorporates a screw-driven insert adjustment in which a rotatable dial assembly (114) (which may include a decorative stone or bezel) is coupled to a threaded screw (116). The threaded screw (116) extends axially through a central threaded bore in the ring body (102) and engages a crescent-shaped insert (118) positioned along an arc of the finger opening (104). A retaining base or screw cap (119) affixes to the lower end of the screw (116) to captively retain the insert (118) while permitting axial motion.

Rotation of the dial (114) in a first direction advances the screw (116) so that the crescent insert (118) projects further inward into the finger opening (104), reducing the effective inner diameter; rotation in the opposite direction retracts the insert (118), enlarging the opening (104). The axial translation of the insert (118) preserves a substantially circular inner profile because the insert subtends only a segment of the circumference rather than producing a stepped radial lip. The insert (118) upper surface presents a smooth contact arc to the wearer and the insert underside may include a low-friction liner (138) to reduce wear and ensure smooth travel.

Materials for the screw-driven embodiment are selected to ensure electrical isolation and mechanical robustness. The ring body (102) and insert (118) may be formed from high-performance insulating materials such as glass-filled PEI, PEEK, alumina or zirconia ceramic, or carbon-fiber filled resin. If enhanced thread strength is required, a metallic element may be employed only when fully encapsulated by a dielectric coating; otherwise polymeric threaded components molded from filled PEEK or acetal are preferred. Thread geometry and tolerances are manufactured to provide desirable frictional self-locking; optional micro-detents (140) or a miniature wave spring (142) may be included to give discrete tactile steps during adjustment.

The internal electronics module (106) resides in a dedicated cavity within the ring body (102) opposite the insert (118) so as to balance mass and avoid interference with the adjustment travel; the electronics module (106) includes the biometric sensor (108), a rechargeable energy source (308), and a wireless communication interface (110). Optical sensors (108) such as LEDs and photodiodes are aligned to the inner skin surface and in some variants are located beneath a transparent portion of the dial (114) or under an optical window. Electrical connection between any rotating dial (114) and the fixed electronics is provided by flexible interconnects (136) or micro spring contacts, dimensioned for the limited rotation range.

Structural parameters for the screw-driven mechanism include insert arc length, travel stroke, and thread pitch. In typical implementations the crescent insert (118) subtends an arc in the range of about 120°-180° and the axial stroke of the screw (116) is selected to adjust the finger-opening diameter (104) across a target range (e.g., about 16 mm to 22 mm internal diameter). Low friction liners (138) and precision molded bearing surfaces ensure smooth operation and long cycle life.

In one embodiment of the adjustable smart ring (100), the segmented gear-drive assembly (126) provides smooth, continuous resizing through an external rotatable dial (130) positioned on the outer surface of the ring body (102). The dial (130) mechanically couples to two or more circumferential segments (127) of the ring that collectively define the finger opening (104). Each segment (127) carries a set of rack teeth (132) on its inner face that mesh with a gear mechanism (130a-130d) beneath the dial. When the dial (130) is rotated, the gear engagement drives the segments (127) radially inward or outward, thereby increasing or decreasing the effective diameter of the finger opening (104) while maintaining a continuous circular profile and uniform pressure around the finger. This configuration performs the same function as a hinge-and-pin connection in other embodiments but eliminates hinge gaps, producing a fully sealed, non-conductive outer surface that enhances both appearance and safety.

In one implementation, the dial (130) supports a pinion gear (130a) coaxially mounted on its shaft and directly meshing with the rack teeth (132) formed on opposing circumferential segments (127). Rotation of the dial (130) causes the pinion (130a) to advance the racks (132) symmetrically in opposite directions, resulting in a uniform expansion or contraction of the ring's inner diameter. The arrangement provides fine, repeatable size adjustment and mechanical self-locking due to friction between the gear teeth, requiring no separate latch. The compact coaxial design also preserves the ring's smooth external geometry, allowing the dial to appear as an ornamental feature or stone mount.

In one embodiment, the segmented gear-drive assembly (126) provides continuous, reversible adjustment of the ring's size through a rotatable dial (130) positioned on the outer surface of the ring body (102). The dial engages the rack teeth (132) of the circumferential segments (127), which are guided to move radially relative to one another. Rotation of the dial results in smooth expansion or contraction of the finger opening (104), maintaining a circular inner profile and uniform pressure distribution. This configuration performs the same function as a hinge-and-pin connection in other embodiments but eliminates hinge gaps, producing a sealed, non-conductive exterior suitable for use around electrical equipment.

In one version, the gear arrangement is configured so that rotation of the dial drives the rack teeth directly through a compact gear train, allowing the user to make fine, incremental size adjustments with minimal torque. The arrangement provides mechanical self-locking due to the frictional engagement of the gears and the close tolerance of the segments, preventing unintended movement once the desired fit is reached. The dial may be recessed or blended with the outer contour of the ring to preserve its aesthetic appearance.

In a further refinement, the gear-drive assembly employs a high-reduction transmission such as a worm or screw interface to achieve finer resolution and to resist back-driving under external forces. This enables the ring to retain its selected fit even when subjected to vibration, impact, or thermal expansion without requiring continuous power or manual tightening. The entire drive system is enclosed within the non-conductive structure of the ring to prevent user contact with any moving or conductive parts.

To avoid accidental operation, the dial may include a spring-loaded locking feature that requires the wearer to pull or press the dial to release the lock before rotation. The lock automatically re-engages when released, preventing unintentional tightening or loosening during daily activities. The dial and the surrounding ring body are constructed entirely from non-conductive materials, such as high-strength polymer or ceramic composites, ensuring electrical insulation. Within the ring body are housed the electronic modules (106), including the biometric sensor (108) and wireless-communication interface (110), all sealed and isolated from the mechanical adjustment system. This integration allows the wearer to monitor physiological parameters and perform contactless transactions while benefiting from a safe, adjustable, and comfortable fit.

In an embodiment illustrated in FIG. 1B the ring (100) embodies a hinged dual-half assembly (120). The ring band is formed from first and second arcuate halves (120a, 120b) joined at one end by a hinge pin (121) and overlapping at the opposite (free) ends. The overlapping region includes complementary interlocking features—curved grooves and mating ridges (128, 129)—that preserve a continuous circular inner surface during relative sliding. A locking latch (122) or a spring-biased detent pin (124) housed in a jacket module secures the overlap at the selected position.

To adjust fit the wearer withdraws the spring-biased pin (124) or unlocks the latch (122), repositions the overlap so as to increase or decrease the inner circumference, and releases the pin or latch to lock the new size. The jacket module that contains the pin (124) provides convenient space for the electronics module (106) (for example the rechargeable cell (308), PCB and sensor array (108)) and may be sealed for environmental protection. The hinge (121) and pin (124) are formed of high-strength non-conductive materials (e.g., ceramic pin and PEEK bushing) or polymeric composites to maintain electrical isolation.

The overlapping halves (120a,120b) are configured to provide discrete or continuous adjustment. Where discrete indexing is preferred, notches are provided for the detent (124) to engage. Where a continuously variable overlap is desired, interfacing ramps and a friction interface maintain position. The hinge (121) may also be configures to permit a full open position for emergency donning/doffing: when the latch (122) is fully released the ring halves swing open to create a large opening, facilitating removal in the event of swelling or entrapment.

In an embodiment shown in FIG. 1D a segmented gear-drive assembly (126) offers fine and continuous radial adjustment. The ring circumference is formed of two or more circumferential segments (127) carrying inter-meshing grooves (128) and ridges (129) on mating faces to maintain alignment. A low-profile rotatable gear dial (130) with an integrated pinion engages rack teeth (132) formed along the inner edges of the segments (127). Rotation of the gear dial (130) displaces the segments (127) radially, thereby varying the inner diameter.

The gear dial (130) is spring-biased axially to provide a pull-to-unlock action: the user pulls the dial outward against a spring preload to disengage a locking detent (140), rotates the dial to the desired position, and releases the dial to re-engage the detent thereby preventing accidental rotation during normal wear. Gear components (126,130,13l ) are preferably molded from wear-resistant engineering plastics such as acetal (POM), nylon 12, or filled PEEK and may incorporate PTFE-filled bearings to reduce friction. The gear ratio and tooth profile are selected to provide a resolution suitable for fine adjustment (for example 0.1-0.3 mm per detent step or per revolution depending on gearing).

The outer crown formed by the dial (130) and adjacent surfaces are styled to maintain the ring's normal appearance. The drivetrain and segment geometry are sealed by thin elastomeric gaskets to protect against ingress. A small pressure equalization port and dust seal may be incorporated to handle thermal expansion while preserving environmental rating.

All manual adjustment embodiments described herein—including the screw/insert arrangement (114, 116, 118, 119), the hinged dual-half assembly (120, 120a, 120b, 121, 122, 124), and the segmented gear-drive mechanism (126, 127, 128, 129, 130, 132) are configured so that all movable and load-bearing elements remain concealed within the non-conductive envelope of the ring body (102). This concealment ensures that the outer profile of the smart ring (100) retains the aesthetic of a conventional jewellery ring without exposing functional seams, apertures, or metallic parts. The visible exterior is defined entirely by user-touchable components (134), each formed of dielectric materials such as ceramic, glass-filled polymer, or composite resin so that no conductive path exists between any internal conductor and the wearer's skin.

The mechanical interfaces are configured with flush transitions and continuous curvature, allowing the finger to contact only smooth arcs regardless of adjustment state. For example, in the screw-insert version the top of the insert (118) aligns seamlessly with the inner wall of the ring body (102) in both its extended and retracted positions. In the hinged-halves embodiment the overlapping ridges (129) and grooves (128) interlock to form a uniform circular inner contour free of discontinuities, while the gear-segment mechanism maintains precise radial registration so that gaps remain narrower than 0.1 mm, eliminating pinch points or snag hazards.

Each embodiment employs detent features (140), wave springs (142), and low-friction liners (138) at key interfaces to provide smooth operation and tactile feedback. The detents (140) act as micro-indexing stops that define discrete incremental positions during rotation or sliding, preventing drift from vibration or incidental contact. The wave springs (142), typically formed from stainless or polymer spring foil isolated from touch surfaces, apply controlled axial preload to maintain engagement between threads, racks, or latching elements, eliminating play while still permitting fine adjustment with minimal torque. The liners (138) fabricated from PTFE or acetal act as dry-lubricated bearing surfaces to minimize wear and to maintain repeatable motion cycles over the lifetime of the product.

To further enhance the user experience and durability, all mechanical tolerances are balanced between tight fit for water-dust sealing and free movement for smooth actuation. Elastomeric micro-seals may be co-molded around the moving interfaces to achieve ingress protection equivalent to IPX5-IPX7, while maintaining the feel of a luxury accessory. The present disclosure's emphasis is on mechanical integrity with aesthetic continuity: the user perceives only a monolithic ornamental band even though a complex variable-geometry mechanism operates beneath the surface.

These combined measures—concealment of moving elements, dielectric isolation of external surfaces, integrated tactile detents, and low-friction resilient preload—yield a robust architecture that maintains both industrial-safety compliance and premium wear comfort while enabling continuous manual adjustment.

In all embodiments, the electronic module (106) and associated subcomponents are physically and electrically isolated from the mechanical drive assemblies of the adjustment mechanism (112). The module (106) is embedded within a sealed compartment of the non-conductive ring body (102), spaced from any moving part such as the screw (116), hinge (121), or gear assembly (126), by a defined separation barrier wall. This arrangement ensures that mechanical stresses generated during adjustment are not transmitted to delicate electronic circuits. The isolation also maintains the dielectric continuity of the outer structure, preventing any inadvertent conduction path between the internal electronic elements and the user-touchable external components (134).

The electronic module (106) typically includes a biometric sensor (108) such as a photoplethysmographic (PPG) optical sensor with paired light emitters and photodiodes, a microcontroller and communication circuit (110) providing wireless connectivity (Bluetooth, NFC, or other standard protocol), and a rechargeable power source (308). These components are mounted on a miniature rigid-flex printed circuit assembly (136a), enabling both compact placement and tolerance of localized flexing due to ring adjustment. The PCB substrate may be polyimide or liquid crystal polymer to ensure dimensional stability under temperature variation.

The optical path of the biometric sensor (108) is oriented through an aperture or window located on the inner surface of the ring facing the skin. The window may be a transparent sapphire, quartz, or polymer lens that is optically clear but electrically insulating. The geometry of this window is aligned so that the light-emitting diodes and photodiodes maintain constant optical coupling to the finger tissue irrespective of adjustment range, using fixed angles or diffused beam paths. Anti-reflective coatings and light-baffling micro-channels can be molded into the ring body (102) to reduce internal reflections and improve signal-to-noise ratio.

In embodiments where the adjustment mechanism involves rotation of an external dial (114 or 130), electrical continuity between stationary electronics and moving components is achieved through flexible printed interconnects (136) or spring-contact slip interfaces (136b). These interconnects are configured for limited angular displacement and incorporate fatigue-resistant conductors such as rolled-annealed copper with parylene coating. The wiring layout ensures that even at maximum dial rotation, strain levels remain below 30% of allowable elongation, guaranteeing long operational life. In some configurations, non-contact magnetic or optical couplers are used to transmit sensor or actuator signals across rotating boundaries, eliminating the need for physical conductive joints.

To protect the electronics against electromagnetic interference (EMI) and electrostatic discharge (ESD), a thin shielding layer (146) of conductive polymer or carbon-loaded paint may be applied to the internal surfaces of the electronics cavity. This shield is grounded only to internal reference planes within the electronics module and is completely isolated from user-touchable external surfaces (134). The non-conductive materials of the ring body (102) inherently limit stray capacitance and further contribute to immunity from electrical coupling in industrial environments.

The power source (308), typically a micro lithium polymer or solid-state thin-film battery, is thermally isolated from the skin-contact region by an insulating spacer (148). This prevents any perceptible heat transfer during charging or discharge. In addition, the battery and circuit are potted in dielectric encapsulant to provide structural rigidity and waterproofing. Charging may be accomplished through inductive charging coils (150) embedded circumferentially within the ring body (102) or through contactless resonant coupling, eliminating exposed terminals.

The embedded communication interface (110) may include an NFC antenna (152) printed or etched on the inner surface of the ring body, or a compact Bluetooth Low Energy antenna formed by a tuned loop trace. The non-metallic body allows efficient radio performance because no metallic shielding interferes with signal propagation. The antenna is impedance-matched to the communication circuit (110) and is angularly oriented to maintain effective coupling in all wearing positions.

Environmental protection for the electronics compartment is ensured by precision gaskets and ultrasonic or laser welding of enclosure seams. Such configuration may achieve a water resistance rating of at least IPX7 while still permitting adjustment of the mechanical elements. All adhesives and encapsulants used are biocompatible and hypoallergenic, suitable for prolonged dermal contact.

Through the combination of structural segregation, flexible wiring, dielectric shielding, and optimized sensor orientation, the smart ring (100) provides reliable electronic performance alongside mechanical adjustability. The integration allows continuous biometric monitoring and data transmission without compromising the safety, aesthetics, or tactile quality of the ring.

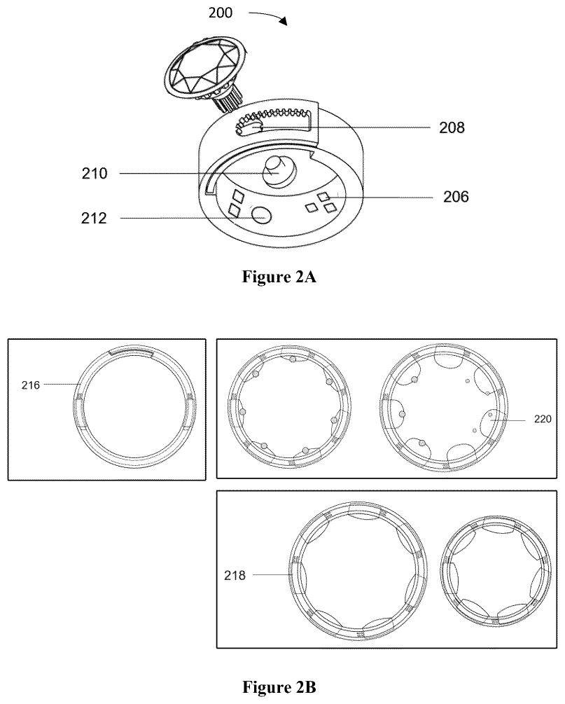

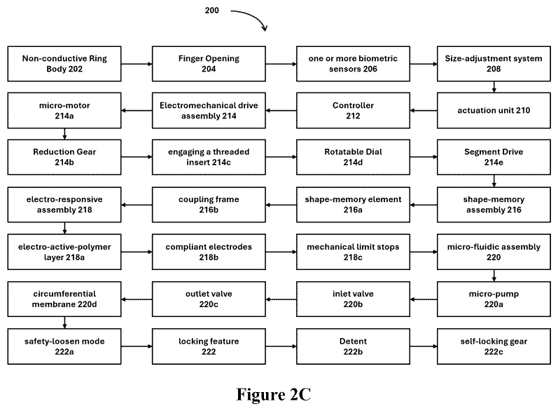

In another embodiment illustrated in FIG. 2A, the adjustable non-conductive smart ring (200) operates through an actuator-assisted mechanical adjuster that enables automatic or semi-automatic variation of the internal diameter of the ring under electronic supervision. The ring (200) comprises a non-conductive annular body (202) defining a finger opening (204); a mechanical size-adjuster (206) which may be of screw-driven, dual-half hinged, or gear-segment type; a compact actuator assembly (208) mechanically coupled to the adjuster; a microcontroller-based fit-controller (210); and at least one biometric sensor (212) arranged to generate physiological data representative of the wearer's condition. The physical integration of these components within the ring body (202) allows the internal diameter to vary dynamically in response to real-time biometric signals while maintaining electrical insulation from the wearer's skin.

The actuator (208) functions as the prime mover for the mechanical size-adjuster (206). It is implemented as a micro-rotary motor of approximately 6 to 8 millimetres outer diameter and less than 3 millimetres axial thickness, or as a miniature linear actuator such as a piezoelectric or voice-coil driver. The output shaft (214) engages a reduction geartrain (216) that transmits torque to the adjustment element with high precision while preventing reverse motion when unpowered. The reduction stage yields mechanical self-locking so that once the actuator halts, the ring maintains the selected diameter without additional energy expenditure. In screw-based embodiments, the actuator (208) interfaces with the screw head (218) through a worm or spur gear; in dial-gear embodiments, it rotates a crown gear (220) that moves the ring segments radially. The actuator housing occupies a sealed cavity within the non-conductive ring body (202), filled with elastomeric dielectric potting that damps vibration, excludes moisture, and isolates every current-carrying component from user-accessible surfaces.

The fit-controller (210) processes incoming signals from the biometric sensors (212) and from an optional array of inner-liner pressure sensors (222) arranged circumferentially along the inner surface of the finger opening (204). The controller executes stored algorithms that compute a fit-quality metric (224) based on one or more parameters such as photoplethysmography amplitude, AC-to-DC ratio, motion-compensated signal-to-noise ratio, or differential pressure distribution. This metric provides a quantitative indicator of sensor-to-skin coupling. The controller compares the calculated metric with a pre-defined reference range representing optimal physiological contact. When deviation beyond tolerance is detected, the controller energises the actuator (208) through driver (226) to vary the diameter until the metric returns within acceptable limits. Upon completion, power to the actuator is interrupted, and the non-back-drivable geartrain (228) mechanically maintains the achieved adjustment, preventing unintended drift during wear.

The driver circuit (226) includes a pulse-width-modulated current regulator and a feedback loop referencing a micro-Hall sensor or a position encoder (230) attached to the actuator shaft (214). The controller (210) continuously supervises torque, displacement, and thermal data to enforce safety constraints. If motor current or reaction torque exceeds a threshold indicative of obstruction or tissue compression, or if temperature sensed by thermal probe (232) approaches a preset upper limit, the system halts movement and initiates a Safety-Loosen operation (234). This operation reverses the actuator through a calibrated step sequence to enlarge the opening sufficiently to relieve circumferential pressure, thereby protecting the wearer from discomfort or vascular constriction. The firmware records each safety event for diagnostic analysis and user notification.

The power-management routine of the controller maintains extremely low energy consumption. The actuator (208) receives power only during brief adjustment cycles, typically below one second per operation. After adjustment, both actuator and driver enter an inactive state, leaving only essential sensor monitoring active. The rechargeable cell (308) therefore supports extended operating life comparable to that of passive smart rings. Scheduled operations such as “Auto-Fit” at ring placement and “Periodic Re-Fit” during long-term wear occur automatically under firmware control, activated by motion detection or periodic timing signals. These functions preserve consistent contact quality under thermal expansion, perspiration, or physical activity variations without user intervention.

In another configuration shown in FIG. 2B, the automatic adjustment is accomplished through shape-memory materials that undergo controlled dimensional change upon electrical excitation. A shape-memory-alloy tendon (240), preferably nickel-titanium wire of 50 to 150 micrometre diameter, is routed circumferentially around the inner wall of the ring body (202) and anchored to tabs (242) at two or more points. When current passes through the wire by way of a driver (244), resistive heating activates its austenitic phase transformation, producing contraction that draws the anchor tabs together and reduces the ring's internal diameter. When the current ceases, the tendon cools and returns to its martensitic state, with a restoring element (246) or elastic deformation of the ring body expanding the opening back to its prior dimension. The tendon resides between thermally insulating layers (248) that restrict heat conduction toward the wearer's skin, ensuring safe operation under all duty cycles.

The driver (244) modulates current through pulse-width control based on temperature feedback from sensor (232) positioned adjacent to the tendon (240). Firmware limits both peak current and duty cycle such that the temperature at the inner surface of the ring remains below approximately 41 degrees Celsius. The SMA actuation delivers a stroke (ΔD) of roughly 0.5 to 1.5 millimetres in ring diameter, sufficient to correct for typical finger-swelling variations encountered in daily use. The transition occurs silently and within fractions of a second, providing imperceptible size compensation and stable contact for biometric sensing. Repeated cycling tests confirm consistent strain recovery without structural fatigue, due to the small amplitude of each transformation.

A further embodiment employs a shape-memory-polymer segment (250) incorporated within the ring circumference. Embedded serpentine resistive heaters (252) raise the temperature of the polymer segment above its glass-transition temperature (Tg≈40-60° C.), causing temporary softening and allowing a micro-linkage (254) to reposition the softened segment to a desired geometry. Once the heaters deactivate and the material cools, the segment re-hardens, locking the new circumference. The heaters are deposited conductive traces encapsulated in dielectric layers (256) that electrically insulate the circuit from the wearer. Thermal feedback from sensor (232) maintains homogeneous temperature distribution across the segment, preventing hot spots. The SMP formulation may include thermoplastic polyurethane, crosslinked epoxy, or hybrid copolymer with tuned Tg for predictable actuation behaviour over repeated cycles.

In another automatic configuration, a dielectric electroactive-polymer (EAP) actuator (260) modulates ring size by voltage-induced strain. The actuator comprises a dielectric elastomer film (262) positioned between compliant electrodes (264) and driven by a high-voltage circuit (266). Application of an electric field causes lateral expansion of the film, translating into a small but precise radial movement of the ring's inner circumference. Mechanical stops (268) define the permissible travel range, ensuring repeatable actuation without over-extension. The EAP structure is fully encapsulated within non-conductive barriers and conformal coatings, isolating high-potential elements from all user-touchable surfaces. The drive voltage, though relatively high, operates at microampere currents, thus inherently safe when encapsulated. The resulting motion offers smooth, continuous fit adjustment in response to electronic control signals with sub-millimetre resolution.

Another version employs a microfluidic membrane system (270) distributed around the inner circumference of the ring body (202). The membrane consists of multiple compliant cells (272) interconnected by micro-channels linked to a miniature pump (274). The pump, actuated by electrostatic or piezoelectric means, introduces or removes fluid to inflate or deflate the cells. Inflation moves the inner wall inward to tighten the ring, while deflation expands the inner diameter. One-way check valves (276) retain pressure without continuous power, permitting the ring to maintain its size indefinitely. A relief port (278) and pressure sensor (280) monitor internal pressure to prevent over-inflation. The controller (210) interprets feedback from the pressure sensor and the fit-quality metric (224) to achieve uniform contact pressure around the circumference by selectively adjusting individual cells.

All automatic embodiments operate under a hierarchical control framework implemented in the controller (210). The firmware includes multiple operational routines:
Auto-Fit adjusts the opening diameter until the computed metric (224) falls within the target range representing optimal biometric coupling.
Safety-Loosen is triggered when swelling indicators such as elevated skin temperature, excessive inner pressure, or abnormal pulse amplitude are detected, initiating controlled expansion by a predefined increment.
Profile Modes such as “Sleep” or “Exercise” adjust baseline fit according to activity levels inferred from accelerometer data.

Digital filters suppress noise in sensor readings, and hysteresis thresholds prevent repetitive cycling. Each actuation instance is time-stamped and stored in non-volatile memory for diagnostics and performance tracking.

Energy for the automatic embodiments derives from the rechargeable cell (308) interfaced with a power-management circuit (282). The circuit incorporates over-current protection, thermal cutoff, and regulated output rails for sensor, logic, and actuator domains. Recharging occurs inductively through a coupling coil (150) embedded in the non-conductive ring wall, enabling sealed construction without external connectors. Typical energy expenditure per adjustment cycle is below 30 milliampere-seconds, imposing negligible reduction in overall runtime. The controller (210) and sensors (212) enter low-power sleep states when motion sensors report inactivity, awakening periodically or upon significant environmental change.

Thermal and mechanical safety are achieved through redundant layers of protection. Each active component is encapsulated within at least two dielectric coatings of cumulative thickness not less than 200 micrometres. The nearest conductive trace is separated from the skin-facing surface by a minimum radial distance of 2 millimetres. Thermal modelling and experimental measurement confirm that surface temperature remains within comfortable physiological limits even under maximum actuation duty. Structural ribs and flexible potting within the ring body (202) distribute mechanical stresses evenly, preventing concentration at material interfaces or actuator mounts.

The non-conductive external surface (134) of the automatic embodiments remains smooth and continuous, free of discontinuities or exposed fasteners. Elastomeric isolation mounts (284) support the actuator housing, attenuating audible vibration and tactile sensation during motion. Internal damping materials further suppress resonance, ensuring silent operation. The wearer perceives no movement or noise while the automatic adjustment process proceeds internally, maintaining the ornamental character of the ring.

The actuator-based embodiments collectively integrate miniature electromechanical systems with embedded sensing and intelligent control within a unified non-conductive structure. These embodiments provide continuous or event-triggered optimization of fit, responsive safety loosening under physiological stress, and retention of size without continuous energy consumption. Through controlled interaction between the actuator, sensors, and controller, the ring achieves automatic adaptability while maintaining mechanical integrity, electrical isolation, and comfort suitable for prolonged industrial or medical usage.

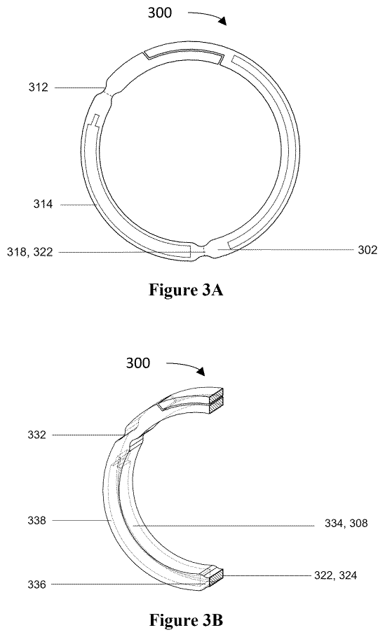

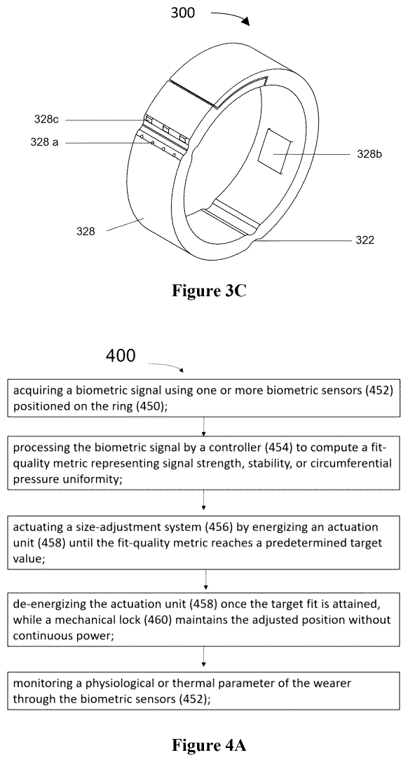

In another embodiment illustrated in FIGS. 3A to 3C, the adjustable smart ring incorporates a safety-release structure that permits controlled separation of the ring body during mechanical overload or emergency removal. The smart ring (300) includes a non-conductive ring body (302) defining a finger opening (304), a safety-release system (312), and an electronic module (306) comprising a power source (308), circuit assembly (310), and communication and sensing elements housed within sealed compartments of the ring body. The safety-release system (312) introduces predetermined structural features that ensure the ring remains intact during ordinary use yet separates along a defined path when exposed to forces that would otherwise endanger the wearer's finger.

The safety-release system (312) is realised through one or more structural interfaces formed in the non-conductive ring body (302). A break-away interface (314) provides mechanical parting when a tensile or torsional load exceeds a calibrated threshold (316). This interface is implemented by forming a localized necked section or shear bridge having reduced cross-section and controlled stress concentration such that separation occurs at a predictable load range, typically between 25 and 60 newtons. The interface geometry includes crack-stop fillets to prevent uncontrolled propagation into adjacent regions of the body. The remainder of the ring structure retains sufficient strength for normal wear and for routine adjustment operations. When subjected to accidental entanglement or swelling of the wearer's finger, the break-away section fractures cleanly, enabling immediate release without injury or electrical hazard.

Another configuration of the safety-release system includes a safe-cut indicator (318) situated on the external surface (320) of the ring body. The indicator is precisely aligned with an underlying sacrificial seam (322) located within the wall thickness of the ring. The seam defines a deliberate plane of weakness formed by a reduced-thickness web (324) having between ten and forty percent of the surrounding wall section. This geometry ensures that, when a cutting tool such as a conventional ring cutter or wire saw is applied along the indicator, the fracture initiates and propagates along the seam with minimal resistance. The seam is aligned with a battery keep-out corridor (326) that maintains spatial clearance between the seam and the embedded energy-storage cell (308). The keep-out corridor provides at least two millimetres of separation from any conductor or battery enclosure, thus preventing accidental puncture or short circuit during emergency removal.

The outer indicator (318) is visually and tactilely distinguishable from the adjacent surface for identification under stress or low-visibility conditions. The indicator may consist of a contrasting inlay line, an engraved groove, or a raised tactile bead discernible by touch. Radiopaque fillers or fluorescent pigments are optionally incorporated so that the indicator remains visible under X-ray or ultraviolet illumination during clinical removal procedures. An internal tool-guide channel (1114) corresponding to the seam geometry assists in positioning the cutting blade precisely along the safe path. This channel is dimensioned to accept the standard cutting discs used in medical ring cutters and to maintain alignment through the thickness of the ring, thereby avoiding tool deviation that might otherwise compromise the embedded circuitry.

The sacrificial seam is formed during manufacture by insert-moulding a lower-strength polymer segment between higher-strength outer segments or by laser scoring a calibrated groove after sintering in ceramic variants. For polymeric rings, the seam may be produced as a living hinge geometry that yields under specific stress, while in ceramic or composite versions, the seam comprises a co-sintered region of distinct grain structure that defines the intended fracture plane. Crack-stop fillets (332) at the seam ends arrest fracture propagation beyond the intended zone. Finite-element analysis and physical testing are performed to validate the threshold load range for fracture, ensuring consistent performance across production batches.

An emergency-guidance subsystem (328) provides additional aid in locating the safe-cut region during emergencies. The subsystem includes a lighting element (328a) positioned adjacent the safe-cut indicator (318) and controlled by the main controller (328b) or a dedicated emergency driver. When a fault condition or emergency signal (330) is detected, the lighting element emits a continuous or pulsed illumination directly above the indicator, visually marking the safe cutting path. The activation may be triggered by user input through a companion device, a predefined gesture sequence, or automatic detection of abnormal temperature, swelling, or battery fault. The controller also supports a vibration cue through an optional haptic actuator (328c), guiding the wearer toward removal in low-light or visually obscured environments.

The arrangement of the power source (308) and circuit conductors (334) within the ring body (302) ensures that all live elements remain isolated from the sacrificial seam (322). The minimum clearance (336) between any electrical trace and the seam plane is maintained throughout manufacture. A non-conductive shield (338) positioned between the seam and the battery enclosure provides additional dielectric protection and mechanical reinforcement. The shield may be a molded ceramic or polymer plate fixed into the ring cavity before sealing. In the assembled configuration, the safe-cut indicator (318) is angularly indexed to a reference datum (340) so that it is diametrically opposed to the energy-storage compartment (342). This geometric registration ensures that any emergency cut proceeds through an inactive section of the ring body, thereby avoiding risk of battery breach or conductor exposure.

The materials used for the ring body and safety-release interfaces are selected for predictable mechanical response and long-term biocompatibility. Typical materials include reinforced engineering polymers such as polyether-ether-ketone (PEEK), high-impact polycarbonate, or alumina-based ceramics. The outer coating is hypoallergenic and resistant to sweat, solvents, and ultraviolet degradation. The embedded electronics are encapsulated within waterproof barriers, achieving a minimum ingress protection rating of IPX7. All user-touchable regions remain electrically insulating, ensuring that even in a fractured or cut condition, no conductive part becomes exposed to the wearer.

In operation, the safety-release and safe-cut mechanisms function as the final protective measure. When excessive circumferential load or swelling occurs, the break-away interface (314) separates along its defined plane, releasing the ring halves. In scenarios where automated loosening or actuator response is unavailable, the wearer or a responder follows the visible indicator (318) and uses any standard ring-removal tool to cut along the safe path. The underlying seam (322) ensures smooth progression of the tool, and the illumination or tactile feedback from the emergency-guidance subsystem (328) facilitates accurate alignment. The entire process allows rapid, controlled removal without generating sparks, without contacting live circuitry, and without risk of thermal injury.

The integration of structural and electronic safeguards transforms the ring into a self-protective wearable suitable for both industrial and medical environments. The multiple release modalities—mechanical fracture, guided cutting, and electronic signalling—provide redundant safety layers. The wearer is thus protected against entrapment, battery expansion, or swelling events, and authorized personnel are provided a clear method for safe removal even under adverse conditions.

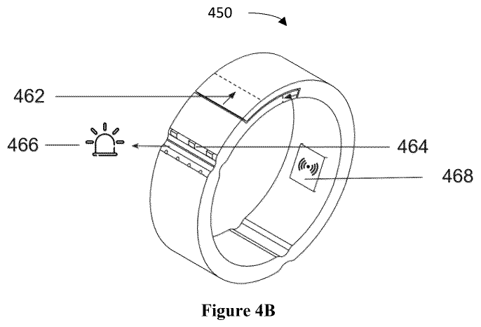

In another embodiment illustrated in FIGS. 4A and 4B, the invention operates as a method (400) executed by an adjustable smart ring system (450) equipped with one or more biometric sensors (452), a controller (454), a size-adjustment system (456), an actuation unit (458), and associated safety-guidance elements (462-468). The method (400) enables continuous monitoring of physiological signals, computation of fit quality, active mechanical or material adjustment of the ring's internal diameter, and automatic release under predetermined safety conditions.

The method (400) begins with acquiring a biometric signal through the sensor array (452) positioned on the inner surface of the ring (450). The sensors (452) include optical photoplethysmography elements, temperature detectors, and optionally a distributed inner-liner pressure array. The signals generated by the sensors (452) are transmitted to the controller (454) for real-time processing. The controller filters noise components and establishes baseline parameters representing the wearer's circulatory and thermal conditions.

The next phase of the method (400) involves processing the acquired signals to compute a fit-quality metric that represents the adequacy of sensor-to-skin contact. The controller (454) evaluates signal amplitude, coherence, and circumferential pressure uniformity. Digital filters suppress motion artefacts, and analytical routines determine the AC/DC ratio of the photoplethysmographic waveform or the variance across the pressure sensors. The resulting metric is compared with a predefined reference range representing optimal physiological coupling between the ring (450) and the wearer's skin.

Once the fit-quality metric is computed, the method (400) proceeds by actuating the size-adjustment system (456) through energisation of the actuation unit (458). The actuator (458) drives a mechanical or material adjuster until the computed fit-quality metric reaches the target value. Depending on the embodiment implemented within the ring (450), the actuator (458) may rotate a micro-motor-driven screw insert, engage a dial-gear system, activate a shape-memory element, energise an electroactive polymer segment, or operate a microfluidic membrane. During adjustment, the controller (454) continuously monitors displacement, drive current, and temperature feedback to ensure safe operation and to prevent excessive tightening or overheating.

After the desired fit is attained, the actuation unit (458) is de-energised, while a mechanical lock (460) retains the adjusted position without further power consumption. The locking mechanism may employ a worm-gear stage, detent feature, or irreversible valve to hold the position passively. The controller (454) records the final fit metric and adjustment parameters as reference data for future recalibration cycles and user-specific adaptation.

During normal wear, the controller (454) remains active in a low-power supervisory mode. It continues to monitor the wearer's physiological or thermal parameters through the sensors (452). Any abnormal deviation—such as elevated surface temperature, reduced perfusion amplitude, irregular photoplethysmography waveform, or excessive inner-liner pressure—triggers an internal comparison process. When the deviation exceeds stored safety thresholds, the controller transitions the system into a protective mode.

Upon entering the protective mode, the controller initiates a safety-loosen operation (462). This operation relieves circumferential pressure by reversing or relaxing the size-adjustment system (456) in controlled increments. The magnitude of reverse movement is determined from calibration data stored in the system memory, ensuring that the release restores normal blood flow without fully disengaging the ring (450). During this process, the controller supervises both torque and temperature feedback, halting the motion immediately if mechanical resistance or temperature rise approaches predetermined limits.

After completion of the safety-loosen sequence, the controller initiates a notification and alert phase. The controller activates a guidance element (464) or generates an alert signal (466) to notify the wearer or an associated monitoring device of the safety event. The guidance element (464) may include a light-emitting diode, haptic actuator, or sound transducer integrated into the ring (450). Each output follows a characteristic sequence—for instance, flashing light or pulsating vibration to indicate that a safety-loosen or emergency state is active and to assist in locating or removing the device.

In emergency situations requiring immediate removal of the ring (450), the controller receives an emergency signal (468) through one of several possible inputs: manual button activation, a command transmitted from a linked mobile device, or automatic fault detection within the system electronics. Upon receiving the emergency signal (468), the controller activates the guidance element (464) to illuminate a safe-cut indicator positioned on the outer surface of the ring (450). The illuminated indicator identifies the underlying sacrificial seam referenced in earlier embodiments, providing a visual and tactile guide for controlled mechanical removal. The illumination pattern may transition from steady to pulsed, and vibration feedback may be emitted to ensure visibility and localization under low-light or stressful conditions.

During and after emergency activation, the controller (454) performs a communication sequence through the integrated wireless module. The sequence transmits diagnostic data, event logs, and biometric records to an external paired device for analysis. The transmitted data establish an operational history, recording both normal adjustments and emergency events for post-incident review or predictive maintenance.

The method (400) further includes energy-management and recovery routines. The controller (454) periodically measures the power cell voltage and internal temperature. When available charge falls below a threshold, the controller suspends adjustment activity and generates a low-energy alert through the guidance element (464). In the event of actuator malfunction, sensor fault, or software error, the controller defaults the ring (450) to an open or relaxed state to eliminate the risk of constriction.

Throughout the operation, the method (400) maintains smooth and incremental actuation to ensure wearer comfort. Each movement of the actuator (458) is verified by real-time feedback from the sensors (452) before continuation, guaranteeing stability, repeatability, and uniform contact pressure. The adjustment is subtle and quiet due to internal damping, ensuring uninterrupted wearability for long durations.

The method (400) achieves a self-regulating, intelligent, and fail-safe operational cycle. The adjustable smart ring (450) continuously maintains optimal biometric sensing performance while automatically protecting the wearer from constriction, overheating, or circulatory restriction. By integrating mechanical, electronic, and algorithmic features within a non-conductive chassis, the system provides autonomous fit optimization, health monitoring, and guided emergency response in a single wearable platform suitable for healthcare, industrial safety, and fintech environments.

The foregoing descriptions of exemplary embodiments of the present disclosure have been presented for purposes of illustration and description. They are not intended to be exhaustive or to limit the disclosure to the precise forms disclosed, and obviously many modifications and variations are possible in the light of the above teachings. The exemplary embodiments were chosen and described in order to best explain the principles of the disclosure and its practical application, to thereby enable others skilled in the art to best utilize the disclosure and various embodiments with various modifications as are suited to the particular use contemplated.

We claim

1. An adjustable smart ring (100) comprising:
a non-conductive ring body (102) wearable on a finger and having a finger opening (104);
one or more electronic modules (106) associated with the ring body (102), the modules including a biometric sensor (108) for monitoring a wearer's physiological parameter and a wireless-communication interface (110) for transmitting data; and
an adjustment mechanism (112) operatively associated with the ring body (102) to selectively vary an effective diameter of the finger opening (104);
wherein the adjustment mechanism (112) mechanically interacts with the ring body (102) to provide continuous and reversible resizing of the finger opening (104) while preserving a continuous circular profile and uniform fit, thereby maintaining non-conductive safety, the adjustment mechanism being implemented through one or more assemblies selected from the group consisting of:
(i) a rotatable dial assembly (114) coupled to a threaded screw (116) and a crescent-shaped insert (118), the dial (114) arranged so that rotation in a first direction advances the insert (118) into the finger opening (104) to reduce its diameter and rotation in an opposite direction retracts the insert (118) to enlarge the opening;
(ii) a hinged dual-half assembly (120) including first and second arcuate halves (120a, 120b) joined by a hinge (121) at one end and overlapping at an opposite end to form a continuous ring shape, a locking latch (122) or spring-biased pin (124) securing the halves (120a, 120b)** in a selectable overlapping position; and
(iii) a segmented gear-drive assembly (126) including two or more circumferential segments (127) having inter-meshing grooves (128) and ridges (129) and a rotatable gear dial (130) engaging rack teeth (132) on the segments (127), rotation of the dial (130) moving the segments (127) radially to increase or decrease the finger-opening diameter;
wherein all user-touchable components (134) are formed of electrically insulating material, rendering the ring (100) safe for use near electrical equipment.

2. The adjustable smart ring (100) of claim 1, wherein the dial (114) includes a decorative element or gemstone holder acting as a user-operable head for the threaded screw (116), the screw being retained by a base (116a) such that the insert (118) moves axially without lateral displacement.

3. The adjustable smart ring (100) of claim 1, wherein the hinged dual-half assembly (120) comprises complementary grooves (128) and ridges (129) maintaining a continuous inner surface across all adjusted positions.

4. The adjustable smart ring (100) of claim 1, wherein the spring-biased pin (124) engages one of multiple detent notches corresponding to discrete size settings.

5. The adjustable smart ring (100) ofclaim 1, wherein the adjustment range provides a diameter variation of at least one and a half millimetres between maximum and minimum positions.

6. The adjustable smart ring (100) of claim 1, wherein the segmented gear-drive assembly (126) includes a rotatable dial (130) disposed on an outer surface of the ring body (102) and engaging the rack teeth (132) of the circumferential segments (127) to move the segments (127) radially, the gear-drive assembly (126) thereby functioning as a replacement for a hinge-and-pin configuration.

7. The adjustable smart ring (100) of claim 6, wherein the gear-drive assembly (126) comprises a pinion gear mounted coaxially with the dial (130) and meshing with the rack teeth (132) formed on the circumferential segments (127) to translate the segments radially inward or outward.

8. The adjustable smart ring (100) of claim 7, wherein the gear-drive assembly (126) further includes a worm-gear drive such that rotation of the dial (130) turns a worm engaging a worm wheel or threaded segment of the ring body (102) to vary the effective diameter of the finger opening (104), the worm gear being non-back-drivable to maintain the selected size without continuous power.

9. The adjustable smart ring (100) of claim 1, wherein the dial (130) incorporates a spring-loaded lock to permit rotation only when pulled outward and to prevent unintentional adjustment when released, and wherein the ring body (102) and dial (130) are formed of non-conductive material enclosing one or more electronic modules (106).

10. An automatically adjustable smart ring (200) comprising:
a non-conductive ring body (202) wearable on a finger and having a finger opening (204);
one or more biometric sensors (206) associated with the ring body (202) for detecting physiological parameters of a wearer;
a size-adjustment system (208) mechanically linked with the ring body (202) to vary an effective diameter of the finger opening (204);
an actuation unit (210) operatively coupled to the size-adjustment system (208) to drive mechanical movement; and
a controller (212) electrically connected to the biometric sensors (206) and the actuation unit (210);
wherein the controller (212) processes signals from the biometric sensors (206) to derive a fit-quality metric and controls the actuation unit (210) until the metric attains a predetermined target;
wherein the size-adjustment system (208) being implemented through one or more assemblies selected from the group consisting of:
(i) an electromechanical drive assembly (214) including a micro-motor (214a) coupled through a reduction gear (214b) engaging a threaded insert (214c), rotatable dial (214d), or segment drive (214e) to effect radial adjustment of the finger opening (204);
(ii) a shape-memory assembly (216) including a shape-memory element (216a) positioned within or around the ring body (202) and mechanically linked to a coupling frame (216b) to contract or expand in response to heating or cooling;
(iii) an electro-responsive assembly (218) including an electro-active-polymer layer (218a) disposed between compliant electrodes (218b) and bounded by mechanical limit stops (218c) to deform under applied voltage; and
(iv) a micro-fluidic assembly (220) including a micro-pump (220a), inlet valve (220b), outlet valve (220c), and a circumferential membrane (220d) inflating or deflating to tighten or loosen the finger opening (204);
wherein the controller (212) enforces temperature and torque limits, activates a safety-loosen mode (222a) when a measured physiological or thermal value exceeds a threshold, and de-energizes the actuation unit (210) after reaching the target fit, while a locking feature (222) including a detent (222b) or self-locking gear (222c) retains the achieved position without continuous power.

11. The automatically adjustable smart ring (200) of claim 10, wherein the controller (212) derives the fit-quality metric from a photoplethysmography (PPG) signal amplitude, AC/DC ratio, or motion-compensated signal-to-noise ratio.

12. The automatically adjustable smart ring (200) of claim 10, wherein the actuation unit (210) comprises a non-back-drivable micro-motor (214a) with worm-gear reduction preventing reverse rotation when de-energised.

13. The automatically adjustable smart ring (200) of claim 10, wherein the shape-memory element (216a) is thermally isolated from a wearer-contacting surface by a dielectric insulation layer (216c).

14. The automatically adjustable smart ring (200) of claim 10, wherein the electro-responsive assembly (218) is encapsulated within non-conductive barrier layers (218d) to prevent user contact with live electrodes.

15. The automatically adjustable smart ring (200) of claim 10, further comprising an inner-liner pressure sensor array (224) providing circumferential pressure feedback to the controller (212).

16. The automatically adjustable smart ring (200) of claim 10, wherein the controller (212) operates in an intermittent power-saving mode, periodically re-activating the biometric sensors (206) to confirm fit quality.

17. A smart ring (300) comprising:
a non-conductive ring body (302) wearable on a finger and having a finger opening (304);
one or more electronic modules (306) housed within the ring body (302), the modules including a power source (308) and circuit components (310); and
a safety-release system (312) associated with the ring body (302) to enable controlled opening or parting of the ring body (302) along a predetermined structural region, thereby permitting removal of the ring from the wearer's finger under mechanical or emergency conditions;
wherein the safety-release system (312) comprises one or more structural features selected from the group consisting of:
(i) a break-away interface (314) that disengages when a tensile or torsional load exceeds a calibrated threshold (316);
(ii) a safe-cut indicator (318) on an outer surface (320) of the ring body (302) aligned with an underlying sacrificial seam (322) having a reduced-thickness web (324) forming a battery keep-out corridor (326), such that a cut along the indicator (318) avoids contact with the power source (308); and
(iii) an emergency-guidance subsystem (328) including a lighting element (328a) positioned adjacent the safe-cut indicator (318) and a controller (328b) linked to the lighting element (328a) to activate illumination upon detection of a fault or upon receipt of an emergency signal (330);
wherein the reduced-thickness web (324) has a thickness between 10 percent and 40 percent of an adjacent wall thickness and includes crack-stop fillets (332) to limit fracture propagation;
wherein electrical conductors (334) and the power source (308) are spaced to maintain a minimum clearance (336) from the sacrificial seam (322) of at least 2 mm, and a non-conductive shield (338) is positioned between the seam (322) and the power source (308); and
wherein the safe-cut indicator (318) is angularly indexed relative to a fixed datum

(340) on the ring body (302) so that the indicator (318) is diametrically opposed to an energy-storage compartment (342) containing the power source (308).

18. The smart ring (300) of claim 17, wherein the break-away interface (314) includes a shearable pin, tear-away strap, or manual quick-release actuator accessible to the wearer, releasing above a calibrated threshold.

19. The smart ring (300) of claim 17, wherein the sacrificial seam (322) further includes a self-locating tool-guide channel for positioning a cutter or wire saw during emergency removal.

20. The smart ring (300) of claim 17, wherein the safe-cut indicator (318) comprises at least one of: a colour-contrasting groove, tactile ridge, directional marking, radiopaque tracer, or fluorescent tracer.

21. The smart ring (300) of claim 17, wherein the emergency-guidance subsystem (328) further includes a vibration element (328c) actuated with the lighting element (328a) to assist location of the seam under low-visibility conditions.

22. The smart ring (300) of claim 17, wherein the shield (338) is formed of resin encapsulant surrounding the power source (308) and conductors (334).

23. A method (400) of operating an adjustable smart ring (450), the method (400) comprising the steps of:
acquiring a biometric signal using one or more biometric sensors (452) positioned on the ring (450);
processing the biometric signal by a controller (454) to compute a fit-quality metric representing signal strength, stability, or circumferential pressure uniformity;
actuating a size-adjustment system (456) by energizing an actuation unit (458) until the fit-quality metric reaches a predetermined target value;
de-energizing the actuation unit (458) once the target fit is attained, while a mechanical lock (460) maintains the adjusted position without continuous power;
monitoring a physiological or thermal parameter of the wearer through the biometric sensors (452);
wherein the controller (454) is operative to monitor the physiological or thermal parameter and detecting variations indicative of swelling, overheating, or circulatory change;
initiating a safety-loosen operation (462) when the monitored parameter exceeds a defined threshold, the safety-loosen operation comprising reversing or relaxing the size-adjustment system (456) to relieve circumferential pressure;
wherein the controller (454) is enforcing torque and temperature limits and automatically triggering the safety-loosen operation (462) when said limits are exceeded;
activating a guidance element (464) or generating an alert signal (466) to indicate the safety-loosen condition or to aid emergency removal of the ring (450);
wherein the guidance element (464) is being activated upon detection of a fault signal (468) or emergency input and illuminating a safe-cut indicator on the ring (450) to assist in locating a sacrificial seam during removal.

24. The method (400) of claim 23, wherein the fit-quality metric is derived from a photoplethysmography (PPG) amplitude, AC/DC ratio, or motion-compensated signal-to-noise ratio.

25. The method (400) of claim 23, wherein the controller (454) enforces torque and temperature limits during actuation and automatically initiates the safety-loosen operation (462) when either limit is exceeded, and optionally adjusts actuation to minimise circumferential pressure variance while maintaining the target fit.

26. The method (400) of claim 23, wherein the size-adjustment system (456) employs one or more of: a screw-driven insert, hinged dual-half overlap, gear-segment drive, shape-memory element, electro-active polymer segment, or micro-fluidic membrane.

27. The method (400) of claim 23, further comprising unlocking a mechanical detent prior to actuation, re-locking after achieving the target fit, detecting a fault or emergency signal (468), and activating the guidance element (464) to illuminate a safe-cut indicator aligned with a sacrificial seam on the ring (450) to facilitate emergency removal.
